(19) United States
(12) Patent Application Publication
(10) Pub. No.: US 2023/0357441 A1
(43) Pub. Date: Nov. 9, 2023

(54) BISPECIFIC CONSTRUCTS FOR EXPANDING T CELLS AND RELATED METHODS

(71) Applicant: SUNNYBROOK RESEARCH INSTITUTE, Toronto (CA)

(72) Inventors: Jean GARIEPY, Toronto (CA); Amanda SPARKES, Stouffville (CA); Esther MATUS, York (CA); Aaron PRODEUS, Waltham, MA (US)

(21) Appl. No.: 18/029,468

(22) PCT Filed: Oct. 14, 2021
(86) PCT No.: PCT/CA2021/051439

§ 371 Date: Mar. 30, 2023

Related U.S. Application Data
(60) Provisional application No. 63/092,410, filed on Oct. 15, 2020.

(51) Int. Cl. C07K 16/46; A61P 37/02

(52) U.S. Cl. CPC C07K 16/468; A61P 37/02; C07K 2317/622; C07K 2317/31

(57) ABSTRACT

Described herein is a bispecific scFv construct agonizing both CD3 and CD28 pathways. Typically, the construct is soluble and activates, expands, and differentiates human T cells ex vivo. Related compositions and methods are also described, such as methods for expanding, activating, and or differentiating T cells ex vivo and methods of treating cancer.

### FIELD

The present invention relates to T cells. In particular, the present invention relates to bispecific constructs for expanding T cells ex vivo as well as related compositions and methods.

### BACKGROUND

Adoptive cell transfer (ACT) is the transfer of cells into a patient. The cells may have originated from the patient or from another individual. The cells are most commonly derived from the immune system with the goal of improving immune functionality and characteristics. In autologous cancer immunotherapy, T cells are extracted from the patient, genetically modified and cultured in vitro and returned to the same patient.

The adoptive transfer of autologous tumor infiltrating lymphocytes (TIL) or genetically re-directed peripheral blood mononuclear cells has been used experimentally to treat patients with advanced solid tumors, including melanoma and colorectal carcinoma, as well as patients with CD19-expressing hematologic malignancies, cervical cancer, lymphoma, leukemia, bile duct cancer and neuroblastoma, lung cancer, breast cancer, sarcoma, melanoma, relapsed and refractory CD19+ B cell malignancies, including B cell acute lymphoblastic leukemia (B-ALL) harboring rearrangement of the mixed lineage leukemia (MLL). The transfer of regulatory T cells has been used to treat Type 1 diabetes and other autoimmune diseases.

Loffler et al. (2000; Blood; 95:2098-2103 and U.S. Pat. No. 7,575,923) describe novel single-chain multifunctional polypeptides comprising at least two binding sites specific for the CD19 and CD3 antigen, respectively. Further provided are polypeptides, wherein the above-described polypeptide comprises at least one further domain, preferably of pre-determined function. Furthermore, polynucleotides encoding said polypeptides as well as to vectors comprising said polynucleotides and host cells transformed therewith and their use in the production of said polypeptides are described. In addition, compositions, preferably pharmaceutical and diagnostic compositions are provided comprising any of the afore-described polypeptides, polynucleotides or vectors. Described is also the use of the afore-mentioned polypeptides, polynucleotides and vectors for the preparation of pharmaceutical compositions for immunotherapy, preferably against B-cell malignancies such as non-Hodgkin lymphoma.

Grosse-Hovest et al. (2003; Eur. J. Immunol.; 33:1334-1340 and U.S. Pat. No. 7,538,196) describes a first bispecific antibody molecule comprising at least one binding site with a variable domain on a light chain (V_{L}) and a variable domain for the T-cell receptor CD-28, linked thereto on a heavy chain (Vₕ). The antibody molecule further comprises at least one binding site with a variable domain on a heavy chain (V_{H}) and a variable domain for a tumour antigen, linked thereto on a light chain (V_{L}). The variable domains on the heavy chains for both specificities are connected to each other by means of a peptide linker. A second bispecific antibody molecule is bivalent for CD-28 and at least monovalent for the tumour antigen.

Despite the existence of bispecific antibodies or beads that engage both CD3 and CD28, a need exists for the development of alternate effective constructs and compositions for expanding T cells, as well as related methods.

### SUMMARY OF THE INVENTION

In accordance with an aspect, there is provided a bispecific scFv construct agonizing both CD3 and CD28 pathways.

In an aspect, the construct is soluble.

In an aspect, the construct activates, expands, and differentiates human T cells ex vivo.

In an aspect, the construct is active at concentrations in the femtomolar range, such as from about 10 to about 500 fM, such as about 170 fM.

In an aspect, the construct promotes the preferential growth of human CD8⁺ T cells over the course of 12 days in comparison to methods involving immobilized anti-CD3 mAb/soluble anti-CD28 mAb or soluble anti CD3/CD28 mAb complexes.

In an aspect, the construct favors the expansion of a CD8⁺ CD27⁺ T cell phenotype.

In an aspect, the anti-CD28 scFv is at the N-terminus of the construct and the anti-CD3 scFv is at the C-terminus of the construct.

In an aspect, the anti-CD3 scFv is at the N-terminus of the construct and the anti-CD28 scFv is at the C-terminus of the construct.

In an aspect, the construct comprises one or more flexible linkers, such as one, two, or three flexible linkers.

In an aspect, the construct comprises a flexible linker between each heavy and light chain domain of each scFv as well as a flexible linker between each scFv.

In an aspect, the construct engages both signals for TCR activation and co-stimulation at a molar ratio of 1:1.

In an aspect, the construct comprises a purification and/or detection tag.

In an aspect, the construct comprises a histidine tag.

In an aspect, the construct comprises or consists of a polypeptide having at least 80% sequence identity to SEQ ID NO:1:

DIVLTQSPASLAVSLGQRATISCRASESVEYYVTSL
MQWYQQKPGQPPKLLIFAASNVESGVPARFSGSGS
GTNFSLNIHPVDEDDVAMYFCQQSRKVPYTFGGGT
KLEIKRGGGGSGGGGSGGGGSQVKLQQSGPGLVTP
SQSLSITCTVSGFSLSDYGVHWVRQSPGQGLEWLG
VIWAGGGTNYNSALMSRKSISKDNSKSQVFLKMNS
LQADDTAVYYCARDKGYSYYYSMDYWGQGTTVTVS
SASTKGPSVFPLAPSSGSGGGGSGGGGSGGGGSDI
KLQQSGAELARPGASVKMSCKTSGYTFTRYTMHWV
KQRPGQGLEWIGYINPSRGYTNYNQKFKDKATLTT
DKSSSTAYMQLSSLTSEDSAVYYCARYYDDHYCLD
YWGQGTTLTVSSVEGGSGGSGGSGGSGGVDDIQLT
QSPAIMSASPGEKVTMTCRASSSVSYMNWYQQKSG
TSPKRWIYDTSKVASVPYRFSGSGSGTSYSLTISS
MEAEDAATYYCQQWSSNPLTFGAGTKLELKHHHHH
HH

or a fragment thereof.

In an aspect, the construct comprises or consists of a polypeptide having at least 85, 90, 95, 96, 97, 98, or 99% identity to SEQ ID NO:1, or a fragment thereof.

In an aspect, the construct comprises or consists of a polypeptide having SEQ ID NO:1.

In accordance with an aspect, there is provided a polynucleotide encoding the construct described herein.

In an aspect, the polynucleotide comprises or consists of a polynucleotide having at least 80% sequence identity to SEQ ID NO:2:

gacatcgtgctgacacagagccctgcttctctggcc
gtgtctctgggacagagagccaccatcagctgtag
agccagcgagagcgtggaatattacgtgaccagcc
tgatgcagtggtatcagcagaagcctggccagcct
cctaagctgctgatcttcgccgccagcaatgtgga
aagcggagtgcctgccagattttccggctctggca
gcggcaccaacttcagcctgaacattcaccccgtg
gacgaggacgacgtggccatgtacttttgccagca
gagcagaaaggtgccctacacctttggcggaggca
ccaagctggaaatcaagagaggtggcggaggatct
ggcggcggaggaagcggaggcggoggatctcaagt
gaaactgcagcagtctggccctggcctggtcacac
cttctcagagcctgagcatcacctgtaccgtgtcc
ggctttagcctgagcgattacggcgtgcactgggt
ccgacagtctccaggacaaggactggaatggctgg
gagtgatttgggctggcggagggacaaactacaac
agcgccctgatgagccggaagtccatcagcaagga
caacagcaagagccaggtgttcctgaagatgaact
ccctgcaggccgacgacaccgccgtgtactattgc
gccagagacaagggctacagctactactacagcat
ggactactggggccagggcaccaccgtgacagtta
gctctgcctctacaaagggccccagcgtgttccct
ctggctccttctagttctggaagtggcggtggtgg
atcaggcggtggcggttctggcggaggcggaagtg
atattaagctgcagcagagcggagccgagctggct
agacctggtgcctctgtgaagatgagctgcaagac
cagcggctacaccttcaccagatacaccatgcatt
gggtcaagcagcggcctggacagggacttgagtgg
atcggctacatcaaccccagccggggctacaccaa
ctacaaccagaagttcaaggacaaggccacactga
ccaccgacaagagcagcagcacagcctacatgcag
ctgagcagcctgaccagcgaagatagcgccgtgta
ttactgtgcccggtactacgacgaccactactgcc
tggattattggggacagggaacaaccctgaccgtg
tctagtgtggaaggtggcagtggcggtagcggtgg
ctctggtggaagcggcggagtggatgatatccagc
tgactcagtcccctgccatcatgtctgctagccct
ggcgagaaagtgaccatgacctgcagagccagcag
ctccgtgtcctacatgaactggtatcaacaaaaga
gcggcacaagccccaagcggtggatctacgataca
agcaaggtggccagcggcgtgccctatagattttc
tggaagcggatccggcaccagctactccctgacaa
tcagcagcatggaagccgaggatgccgccacctac
tactgccaacagtggtccagcaatcccctgacctt
tggagccggaacaaagctggaactgaagcaccacc
accatcaccac

or a fragment thereof.

In accordance with an aspect, there is provided a vector comprising the polynucleotide described herein.

In accordance with an aspect, there is provided a host cell comprising the vector.

In accordance with an aspect, there is provided a composition comprising the construct described herein and a carrier.

In an aspect, the construct, the polynucleotide, the host cell, or the composition is for adoptive T cell therapy.

In an aspect, the construct, the polynucleotide, the host cell, or the composition is for expanding T cells ex vivo.

In an aspect, the construct, the polynucleotide, the host cell, or the composition is for treating and/or preventing cancer.

In accordance with an aspect, there is provided a method for expanding, activating, and or differentiating T cells ex vivo, the method comprising incubating the T cells with the construct described herein.

In an aspect, the construct is used at concentration of from about 10 to about 500 fM, such as about 170 fM.

In an aspect, the incubating is for a period of about 1, 2, 3, 4, 5, 6, 7, 8, 9, 10, 11, 12, 13, or 14 days, such as about 12 days.

In accordance with an aspect, there are provided T cells made by the method described herein.

In accordance with an aspect, there is provided a method for treating cancer, the method comprising administering the T cells described herein to a subject in need thereof.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIG. 1. Design and purification of T-CEP. (A) Diagram depicting the structure of T-CEP, a T cell expansion protein. T-CEP is a bispecific agent composed of a N-terminal CD28-targeting scFv connected by a short flexible linker (Linker 2) to a CD3-binding scFv. Both scFvs are further defined as linear assemblies of variable heavy (VH) and variable light (VL) chains. A histidine tag (6×His) was inserted at the C-terminus of T-CEP for purification and detection purposes (B) Recombinant T-CEP was produced in HEK 293 cells (Expi293 expression system) and purified by Ni-NTA affinity chromatography. Its purity was confirmed by SDS-PAGE (left panel; Coomassie staining) and by Western blot (right panel; detected using an anti-His tag antibody). The protein migrates as a ˜60 kDa band.

FIG. 2. Characterization of T-CEP binding to human CD3 and human CD28 extracellular domains. (A) Surface plasmon resonance (SPR) sensorgrams depicting the binding of T-CEP to recombinant human FC-tagged CD3ε/δ and recombinant human Fc-tagged CD28 over a range of T-CEP concentrations. (below) Table summarizing T-CEP binding kinetics parameters calculated from SPR sensorgrams. The association rate constant (kₐ), dissociation rate constant (k_{d}) and dissociation constant (K_{D}) were calculated using the 1:1 binding model using BIAevaluation software. (B) Enzyme-linked immunosorbent assay (ELISA) confirming the binding of soluble T-CEP to plate-bound recombinant human CD3-Fc or an IgG control (n=3). The CD3-bound T-CEP was subsequently detected using a biotinylated recombinant human CD28-Fc construct followed by a streptavidin-HRP conjugate. *** P<0.0005.

FIG. 3: T-CEP binds to target antigens as demonstrated using an enzyme-linked immunosorbent assay (ELISA). A sandwich ELISA was carried out by coating wells with T-CEP (4 μg/mL) of 96-well high-protein binding plates and incubated with target antigens. Recombinant human CD3s-Fc (4 μg/mL), recombinant human CD28-Fc (4 μg/mL), and an Fc-matched control (4 μg/mL) were detected using anti-higG-HRP conjugate. (n=3). Terms: rhCD3εδ, recombinant human CD3εδ-Fc; rhCD28, recombinant human CD28-Fc; Symbols: *** P<0.0005.

FIG. 4. Low concentrations of T-CEP promote the ex vivo activation and proliferation of human T cells (A) Representative CFSE profiles depicting the proliferation status of human T cells after a 5-day exposure to T cell expanding conditions, without added cytokines. The immobilized anti-CD3 (i αCD3) and soluble anti-CD28 (s αCD28) agonistic mAbs were used at working concentrations of 5 μg/mL and 2 μg/mL respectively while the final concentration of soluble tetrameric antibody complexes (TACs; STEMCELL) in wells was ˜1.5 μg/mL. T-CEP was dispensed into wells to a final concentration of 10 ng/mL. (B) Representative cytokine secretion levels observed at Day 5 for human T cells exposed ex vivo to cell expansion conditions. Terms: TACs, Tetrameric antibody complexes; iαCD3, Immobilized anti-CD3; sαCD28, Soluble anti-CD28. Symbols: * P<0.05; □, T-CEP; ∘, TACs; ⋄, iαCD3⁺sαCD28; Δ, iαCD3.

FIG. 5: Monitoring the ex vivo expansion of activated human T cells over 12 days. (A) 12-day expansion of purified human T cells from three donors. The number of viable cells were counted using a hemocytometer following the staining of dead cells with trypan blue (n=2). (B) Expansion profiles of CD4⁺ and CD8⁺ T cell subsets from PBMCs of three donors. Cell numbers from the T cell expansion were used alongside the percentage of CD4⁺ and CD8⁺ T cells determined by flow cytometry. The fold expansion was determined based on the starting number of CD4⁺ or CD8⁺ T cells. The Day 12 mean T cell subset fold expansion from the three donors, indicate that T-CEP had the highest fold expansion of CD8⁺ T cells, compared to the other stimulation conditions, whereas TACs demonstrated significantly higher CD4⁺ T cell expansion (C) The percentage of activated human CD4⁺ and CD8⁺ T cell populations relative to the total number of viable lymphocytes as defined by flow cytometry following 12 days of culture. Based on the three donors, targeting CD3 and CD28 with T-CEP (p=0.012) or with a combination of immobilized iαCD3 and soluble sαCD28 mAbs (p=0.018) favored the expansion of CD8⁺ human T cells significantly more than the soluble TACs. Terms: TACs, Tetrameric antibody complexes; iαCD3, Immobilized anti-CD3; sαCD28, Soluble anti-CD28. Symbols: * P<0.05; □, T-CEP; ∘, TACs; ⋄, iαCD3+sαCD28; Δ, iαCD3.

FIG. 6. Human T cell activation following stimulation over a 12-day ex vivo expansion period. The presence of surface markers on human T cells from 3 donors was analyzed by flow cytometry. (A) The surface expression of activation markers CD25 and CD38 exposed to individual stimulation conditions. The increase in Median Fluorescence Intensities (MFI)s for the activation markers was calculated by subtracting the MFI at Day 0 before stimulation. MFI values of both activation markers decreased to baseline values by Day 12 for all 3 donors. T-CEP showed the highest average expression of the activation markers, but there was no significant difference between MFI values, and by Day 12 the activation markers returned to pre-treatment levels (B) The co-expression of PD-1 and LAG-3 markers on human T cells peaked by Day 3 and returned to pre-treatment levels by Day 12. There was no significant difference between the co-expression of exhaustion markers at the peak measured time point in CD4⁺ T cells. In CD8⁺ T cells, the expression of these markers was increased on Day 3 in T-CEP-stimulated cells, but returned to baseline values as in the case of other activation methods by Day 9. Terms: TACs, Tetrameric antibody complexes; iαCD3, Immobilized anti-CD3; sαCD28, Soluble anti-CD28. Symbols: * P<0.05; □, T-CEP; ∘, TACs; ⋄, iαCD3⁺sαCD28; Δ, iαCD3.

FIG. 7. T-CEP favors the ex vivo differentiation of T cells towards a less differentiated human CD8⁺ T cell phenotype. (A) Representative cytometric plots highlighting the expression of CD45RA and CD27, and of CD8⁺ CD27⁺ human T cells on Day 12 following their activation and expansion using a designated treatment. (B) Surface expression at Day 12 of CD45RA and CD27 markers as a percentage of CD4⁺ populations from 3 donors. In the CD3⁺ CD4⁺ T cell subset population, T-CEP treated cells display increased levels of CD45RA-CD27⁺ T cells relative to immobilized anti-CD3 (i αCD3) treated cells (p=0.0098). (C) Surface expression at Day 12 of CD45RA and CD27 markers as a percentage of CD8+ populations from 3 donors. T-CEP significantly increases the CD45RA-CD27⁺ population relative to the i αCD3 alone (p=0.0016) or as a combination of i αCD3 and soluble anti-CD28 (s αCD28; p=0.0318). T-CEP also significantly reduces the CD45RA-CD27⁻ population relative to i αCD3 (p=0.0018), i αCD3 and s αCD28 (p=0.0042), or when activated with soluble tetrameric antibody complexes (TACs) (p=0.0185). (D) The percentage of viable CD8⁺ CD27⁺ viable CD3⁺ lymphocytes at Day 12 observed in human T cell samples collected from 3 donors. T-CEP stimulated T cells co-expressing CD8 and CD27 accounted for 60.80% of the total viable lymphocyte population, relative to cells treated with either i αCD3 alone (p=0.0016), i αCD3 and s αCD28 (p=0.0017), and TACs (p=0.0007) (E) CD27 expression on CD8⁺ T cells (ΔMFI) at Day 12 from each treatment modality. The ΔMFI was calculated by subtracting the MFI value of the appropriate isotype control. The subset of CD8⁺ T cells expressing CD27 is significantly higher in T cells stimulated with T-CEP relative to other stimulation methods [i αCD3 (p=0.0008), i αCD3 and s αCD28 (p=0.0010), and TACs (p=0.0059)]. (F) The expansion of the CD8⁺ CD27⁺ T cells over 12 days was substantially greater when treated with T-CEP than with other stimulation conditions [i αCD3 (p=0.002), i αCD3 and s αCD28 (p=0.0095), and TACs (p=0.0085)]. Terms: TACs, Tetrameric antibody complexes; iαCD3, Immobilized anti-CD3; sαCD28, Soluble anti-CD28. Symbols: * P<0.05; ** P<0.005; *** P<0.0005.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Variants” are biologically active constructs, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The constructs described herein may include modifications. Such modifications include, but are not limited to, conjugation to an effector molecule. Modifications further include, but are not limited to conjugation to detectable reporter moieties. Modifications that extend half-life (e.g., pegylation) are also included. Proteins and non-protein agents may be conjugated to the constructs by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

“Active” or “activity” for the purposes herein refers to a biological and/or an immunological activity of the constructs and/or T cells described herein, wherein “biological” activity refers to a biological function (either inhibitory or stimulatory) caused by the constructs and/or T cells.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the constructs described herein is, in aspects, sufficient to activate, expand, and/or different T cells. In another example, administration of a therapeutically effective amount of the T cells described herein is, in aspects, sufficient to treat and/or prevent cancer or an autoimmune disease.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The constructs and/or T cells described herein may, in aspects, be administered before, during or after treatment with conventional therapies for the disease or disorder in question, such as cancer.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

### T-CEP

Described herein are bispecific scFv constructs. These constructs agonize both the CD3 and the CD28 pathways. Typically, the constructs comprise an anti-CD3 scFv and an anti-CD28 scFv, fused to one another directly or indirectly.

Typically, the construct is small and soluble and activates, expands, and/or differentiates human T cells ex vivo at concentrations in the femtomolar range. For example, the construct is typically active at concentrations of from about 10 to about 500 fM, such as from about 10, about 15, about 25, about 50, about 75, about 100, about 150, about 200, about 250, about 300, about 350, about 400, or about 450 to about 15, about 25, about 50, about 75, about 100, about 150, about 200, about 250, about 300, about 350, about 400, about 450, or about 500 fM. For example, the construct is typically active at concentrations from about 150 to about 200 fM, such as about 170 fM.

The construct described herein typically promotes the preferential growth of human CD8⁺ T cells over the course of about 8 to about 16 days, such as about 12 days, in comparison to methods involving immobilized anti-CD3 mAb/soluble anti-CD28 mAb or soluble anti CD3/CD28 mAb complexes.

In aspects, the construct described herein favors the expansion of a CD8⁺ CD27⁺ T cell phenotype.

It will be understood that the anti-CD28 scFv may be at or near the N-terminus or the C-terminus of the construct and the anti-CD3 would be at or near the corresponding C-terminus or N-terminus. Typically, however, the anti-CD28 scFv is at or near the N-terminus of the construct and the anti-CD3 scFv is at or near the C-terminus of the construct.

Typically, the construct comprises one or more flexible linkers. For example, flexible linkers may be included between the heavy and light chains of the anti-CD28 scFV and/or the anti-CD3 scFv. Additionally or alternatively, there may be a linker between the anti-CD28 scFv and the anti-CD3 scFv. Typically, the construct comprises one, two, three linkers, typically three linkers. The construct typically further comprises a detection tag, such as a histidine tag, which is normally at the C-terminus of the construct.

Typically, the construct engages both signals for TCR activation and co-stimulation at a molar ratio of 1:1.

For example, typically, the construct has the following sequence: DIVLTQSPASLAVSLGQRATISCRASESVEYYVTSLMQWYQQKPGQPPKLLIFAASNVESGVPARFSGSG SGTNFSLNIHPVDEDDVAMYFCQQSRKVPYTFGGGTKLEIKR-OPTIONAL LINKER-QVKLQQSGPGLVTPSQSLSITCTVSGFSLSDYGVHWVRQSPGQGLEWLGVIWAGGGTNYN SALMSRKSISKDNSKSQVFLKMNSLQADDTAVYYCARDKGYSYYYSMDYWGQGTTVTVSSASTKGPSV FPLAPS-OPTIONAL LINKER-DIKLQQSGAELARPGASVKMSCKTSGYTFTRYTM HWVKQRPGQGLEWIGYINPSRGYTNYNQKFKDKATLTTDKSSSTAYMQLSSLTSEDSAVYYCARYYDD HYCLDYWGQGTTLTVSSVE-OPTIONAL LINKER-VDDIQLTQSPAIMSASPGEKV TMTCRASSSVSYMNWYQQKSGTSPKRWIYDTSKVASVPYRFSGSGSGTSYSLTISSMEAEDAATYYCQ QWSSNPLTFGAGTKLELK-OPTIONAL DETECTION TAG
where regular font represents the sequence for anti-CD28 and italic font represents the sequence for anti-CD3; underlining represents the linkers, if present, and bolding represents a detection tag, if present.
For example, the construct typically has the following sequence:

DIVLTQSPASLAVSLGQRATISCRASESVEYYVTS
LMQWYQQKPGQPPKLLIFAASNVESGVPARFSGSG
SGTNFSLNIHPVDEDDVAMYFCQQSRKVPYTFGGG
TKLEIKRGGGGSGGGGSGGGGSQVKLQQSGPGLVT
PSQSLSITCTVSGFSLSDYGVHWVRQSPGQGLEWL
GVIWAGGGTNYNSALMSRKSISKDNSKSQVFLKMN
SLQADDTAVYYCARDKGYSYYYSMDYWGQGTTVTV
SSASTKGPSVFPLAPSSGSGGGGSGGGGSGGGGSD
IKLQQSGAELARPGASVKMSCKTSGYTFTRYTMHW
VKQRPGQGLEWIGYINPSRGYTNYNQKFKDKATLT
TDKSSSTAYMQLSSLTSEDSAVYYCARYYDDHYCL
DYWGQGTTLTVSSVEGGSGGSGGSGGSGGVDDIQL
TQSPAIMSASPGEKVTMTCRASSSVSYMNWYQQKS
GTSPKRWIYDTSKVASVPYRFSGSGSGTSYSLTIS
SMEAEDAATYYCQQWSSNPLTFGAGTKLELKHHHH
HH

Sequences that are substantially identical to the above sequences are also contemplated, such as those that are at least about 80, 81, 82, 83, 84, 85, 86, 87, 88, 89, 90, 91, 92, 93, 94, 95, 96, 97, 98, or 99% identical. Fragments of the sequences or the substantially identical variant sequences are also contemplated herein.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The constructs described herein may also comprise additional sequences to aid in their expression, detection or purification. Any such sequences or tags known to those of skill in the art may be used. For example, and without wishing to be limiting, the constructs may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the constructs may be linked to a cargo molecule; the constructs may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; an anti-cancer antibody for immunotherapy; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the construct may be linked to a fluorescent agent such as FITC or may genetically be fused to the Enhanced Green Fluorescent Protein (EGFP).

The construct described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, 10⁻⁹ M, 10-10 M, 10-11 M, or 10-12 M.

Also described herein are nucleic acid molecules encoding the constructs described herein, as well as vectors comprising the nucleic acid molecules and host cells comprising the vectors.

Polynucleotides encoding the constructs described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

For example, a polynucleotide encoding the constructs described herein in aspects has the sequence:

gacatcgtgctgacacagagccctgcttctctggc
cgtgtctctgggacagagagccaccatcagctgta
gagccagcgagagcgtggaatattacgtgaccagc
ctgatgcagtggtatcagcagaagcctggccagcc
tcctaagctgctgatcttcgccgccagcaatgtgg
aaagcggagtgcctgccagattttccggctctggc
agcggcaccaacttcagcctgaacattcaccccgt
ggacgaggacgacgtggccatgtacttttgccagc
agagcagaaaggtgccctacacctttggcggaggc
accaagctggaaatcaagagaggtggcggaggatc
tggcggcggaggaagcggaggcggcggatctcaag
tgaaactgcagcagtctggccctggcctggtcaca
ccttctcagagcctgagcatcacctgtaccgtgtc
cggctttagcctgagcgattacggcgtgcactggg
tccgacagtctccaggacaaggactggaatggctg
ggagtgatttgggctggcggagggacaaactacaa
cagcgccctgatgagccggaagtccatcagcaagg
acaacagcaagagccaggtgttcctgaagatgaac
tccctgcaggccgacgacaccgccgtgtactattg
ogccagagacaagggctacagctactactacagca
tggactactggggccagggcaccaccgtgacagtt
agctctgcctctacaaagggccccagcgtgttccc
tctggctccttctagttctggaagtggcggtggtg
gatcaggcggtggcggttctggcggaggcggaagt
gatattaagctgcagcagagcggagccgagctggc
tagacctggtgcctctgtgaagatgagctgcaaga
ccagcggctacaccttcaccagatacaccatgcat
tgggtcaagcagcggcctggacagggacttgagtg
gatcggctacatcaaccccagccggggctacacca
actacaaccagaagttcaaggacaaggccacactg
accaccgacaagagcagcagcacagcctacatgca
gctgagcagcctgaccagcgaagatagcgccgtgt
attactgtgcccggtactacgacgaccactactgc
ctggattattggggacagggaacaaccctgaccgt
gtctagtgtggaaggtggcagtggcggtagcggtg
gctctggtggaagcggcggagtggatgatatccag
ctgactcagtcccctgccatcatgtctgctagccc
tggcgagaaagtgaccatgacctgcagagccagca
gctccgtgtcctacatgaactggtatcaacaaaag
agcggcacaagccccaagcggtggatctacgatac
aagcaaggtggccagcggcgtgccctatagatttt
ctggaagcggatccggcaccagctactccctgaca
atcagcagcatggaagccgaggatgccgccaccta
ctactgccaacagtggtccagcaatcccctgacct
ttggagccggaacaaagctggaactgaagcaccac
caccatcaccac

or a sequence at least 80% identical thereto, or a fragment thereof.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The constructs described herein can be expressed in cell lines other than in hybridomas. Nucleic acids, which comprise a sequence encoding a polypeptide, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The constructs described herein can be fused to additional amino acid residues. Such amino acid residues can be a peptide tag to facilitate isolation, for example. Other amino acid residues for homing of the antibodies to specific organs or tissues are also contemplated.

Also described herein are methods for expanding, activating, and or differentiating T cells ex vivo. The methods comprise incubating the T cells with the construct described herein for a period of time, for example about 1, 2, 3, 4, 5, 6, 7, 8, 9, 10, 11, 12, 13, or 14 days, such as about 12 days. The construct may be used at any concentration but is typically used at a concentration of from about 10 to about 500 fM, such as about 170 fM.

Also described herein are T cells made by the method above. These T cells may be used for any desired purpose but are typically used for research, for treating and/or preventing cancer, and/or for treating and/or preventing an autoimmune disease.

Any suitable method or route can be used to administer the constructs and T cells described herein. Routes of administration include, for example, oral, intravenous, intraperitoneal, subcutaneous, or intramuscular administration.

It is understood that the constructs or T cells described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way in the remainder of the disclosure.

### EXAMPLES

### Example 1: T-CEP: A Soluble T Cell Activator that Favors the Ex Vivo Expansion of Multifunctional Human CD8⁺ CD27⁺ T Cells

### Abstract

Adoptive cell therapy involves the infusion of tumor-reactive T cells into cancer patients to provide antitumor immunity. The ex vivo expansion and differentiation of such T cells are key parameters that affect their therapeutic potential. Human T cells are presently expanded in culture through the use of anti-CD3 and anti-CD28 monoclonal antibodies immobilized on beads, expressed on cells or assembled in the context of soluble antibody complexes. Here we report the design of a small, bispecific scFv construct agonizing both CD3 and CD28 pathways. This soluble T cell expansion protein, termed T-CEP, activates, expands and differentiates human T cells ex vivo at concentrations in the femtomolar range. Importantly, T-CEP promotes the preferential growth of human CD8⁺ T cells over the course of 12 days in comparison to methods involving immobilized anti-CD3 mAb/soluble anti-CD28 mAb or soluble anti CD3/CD28 mAb complexes. The differentiation profile of the resulting human T cell population is also singularly impacted by T-CEP, favoring the expansion of a preferred CD8⁺ CD27⁺ T cell phenotype. The activity profile of T-CEP on human T cells ex vivo suggests its use in generating human T cell populations that are more functionally suited for adoptive cell therapies.

### INTRODUCTION

Adoptive cell therapy (ACT) regroups cell-based clinical approaches typically exemplified by lymphocytes with antitumor activity being transferred into patients to eliminate specific cancer cells.¹ Such approaches have been successful in treating hematological malignancies, metastatic melanoma, and metastatic breast cancer.²⁻⁴ Depending on the type of malignancy, treatment may be carried out using a patient's own tumour infiltrating lymphocytes (TILs), or by designing chimeric antigen receptor (CAR) T cells, or by employing genetically-modified T cells. These lymphocytes and in particular highly functional human T cells, must all be expanded ex vivo before infusion into a patient⁵⁻⁷; a key step that requires overcoming challenges associated with expanding such cells in order to generate clinically effective doses.⁷ Important steps for the ex vivo activation, expansion, and survival of human T cells include a T cell receptor (TCR)-mediated activation signal, a co-stimulatory signal, and signalling through cytokines.⁸ In vitro polyclonal T cell activation is generally accomplished through the use of agonistic antibodies targeting CD3 and CD28 providing both TCR activation and co-stimulatory signals respectively, with appropriate cytokines being supplemented.⁵

There are several clinically important criteria linked to the success of using human T cells for ATC. First, CD8⁺ T cells are favored in ACT due to their cytolytic activity 9, although transferred CD4⁺ T cells also display the potential to enhance CD8⁺ T cell-mediated tumor rejection.¹⁰ A second key parameter is the differentiation state of the resulting T cell product expanded ex vivo. Specifically, T cell differentiation events take place during their expansion which can ultimately lead to a loss of crucial ACT characteristics required for their optimal persistence and function in vivo.^{11,12} Less differentiated T cells are more desirable, which can be distinguished based on the surface expression of known markers for naïve T cells (Tn), stem cell memory (Tscm), and central memory (Tcm) T cells, as opposed to more differentiated effector memory (Tem), and terminally-differentiated T cells (Temra).^{13,14} T cell activation, proliferation, and differentiation through agonizing both CD3 and CD28 pathways in vitro can expand cells in a unique manner based on how both signals are presented to human T cells and on the cytokines that are provided. Studies have now shown that different expansion methods and the modification of such methods can lead to T cells displaying more desirable phenotypes.¹⁵⁻¹⁷ Parameters such as ligand density¹⁸, mechanical forces^{19,20}, strength of the interaction²¹, and duration of the signal that activates human T cells affects the outcome of the ATC response.²² Triggering T cell activation through different formats of signal delivery is therefore expected to skew the final T cell phenotype.

In this report, we have engineered a highly potent, monomeric protein that expands human T cells ex vivo when added as a soluble factor at low concentrations. The potency of this T cell expansion protein (termed T-CEP) was compared to an established protocol where an immobilized agonistic anti-CD3 mAb is combined with a soluble anti-CD28 mAb. T-CEP was also compared to a commercially available soluble T cell activator that targets both human CD3 and CD28 in the format of a combined solution of monospecific tetrameric antibody complexes (TACs). T-CEP was shown to engage both CD3 and CD28 on human T cells which lead to a high level of proliferative activity. Furthermore, T-CEP demonstrated an improved ability to expand CD8⁺ human T cell populations exhibiting a less differentiated T cell phenotype that have been clinically linked to more successful ATC outcomes in patients.

### Results

### Design and Production of T-CEP

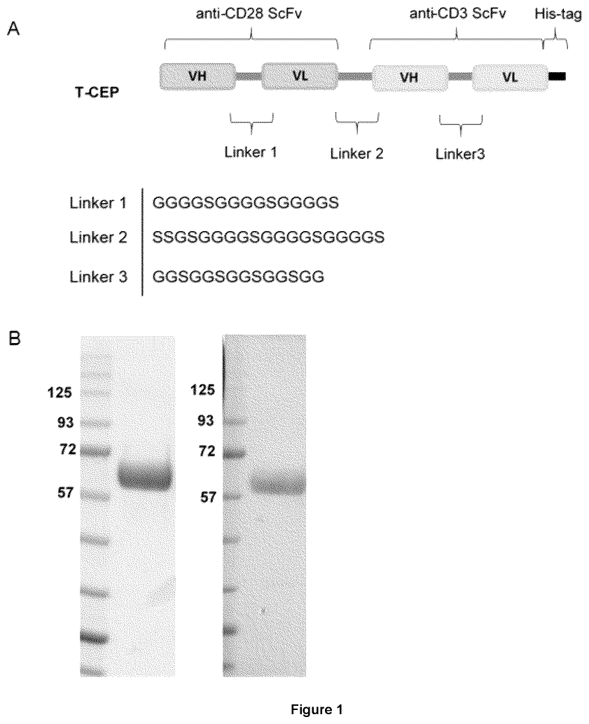

A 60 kDa soluble T cell expansion protein (T-CEP) was designed by fusing together two distinct single chain variable fragments (scFvs) that respectively bind to human CD3 and human CD28 and act as agonists in activating both signalling pathways (FIG. 1A). These selected scFv sequences have independently been shown to agonise CD3²³ and CD28²⁴ respectively, and accordingly activate human T cells. These scFvs were linked together using a short flexible spacer (SSGSGGGGSGGGGSGGGGS), similar to previously described linkers.²⁵ A 6-histidine tag was added at the C terminus of the construct for purification purposes and for detecting the construct (FIG. 1A). Of note, two T-CEP constructs were originally built to assess whether the orientation (i.e., the anti-CD28 scFv at the C-terminus or the N-terminus of the construct) had an impact on their functional properties. The construct as depicted in FIG. 1A, with the scFv targeting CD28 at the N-terminus, and the CD3 scFv placed at its C-terminus was ultimately chosen as the lead compound, due to higher production yields and more potent T cell expansion activity (results not shown). T-CEP was expressed as a secreted, soluble protein produced in HEK293 cells [Expi293 system] and purified using immobilized metal affinity chromatography. Its purity was confirmed by SDS-PAGE and Western blot analysis, migrating as a 60 kDa band, corresponding to the expected molecular weight of T-CEP based on its amino acid sequence (FIG. 1B).

Based on its structure, T-CEP is an agent that is designed to engage both signals for TCR activation and co-stimulation at a molar ratio of 1:1. The other currently available commercial soluble T cell activator (STEMCELL technologies) delivers these signals in the format of soluble monospecific tetrameric antibody complexes (TACs). The TAC is prepared using monoclonal antibodies from one animal towards CD3 or CD28, and mixing them with monoclonal antibodies from a second animal against the Fc-fragment of the antigen targeting antibody.²⁶ The prepared TACs are then combined in one solution at a 1:1 ratio of anti-CD3 TACs and anti-CD28 TACs, resulting in an expected component ratio of 1:1.

### T-CEP Binds to the Human CD3 and CD28 Extracellular Domains

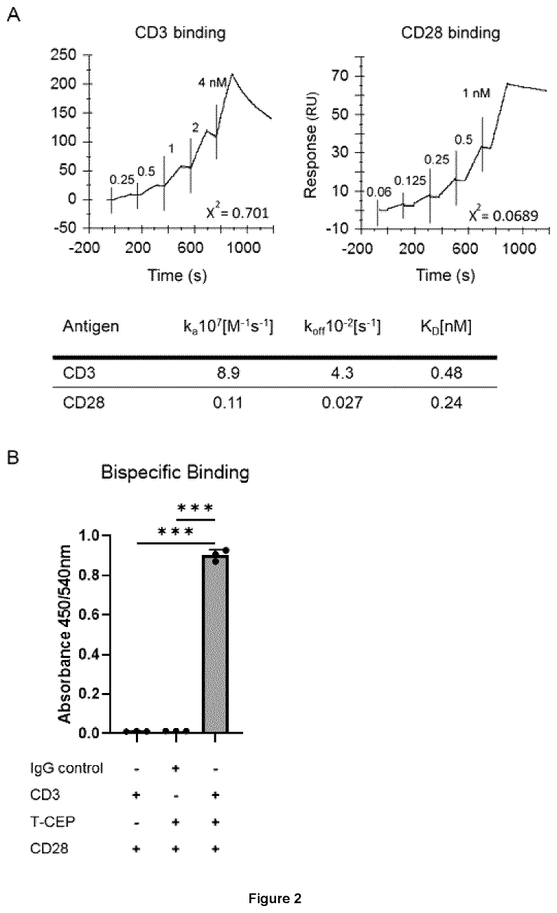

The binding of T-CEP to the extracellular domains of CD3 and CD28 was confirmed by surface plasmon resonance (SPR). Specifically, Fc-tagged recombinant human CD28 and human CD366 proteins were immobilized on a Protein G-modified chip and increasing concentrations of T-CEP were flown over each immobilized target. Resulting sensograms were analyzed (Biacore T200 evaluation software) to calculate binding kinetic parameters (fitted to a Langmuir 1:1 binding model, FIG. 2A). T-CEP was shown to bind tightly to both CD3 and CD28 targets with equilibrium dissociation constants (K_{D})s of 0.48 nM and 0.24 nM respectively. The dissociation rate (k_{off}) of T-CEP binding to CD3 was 4.3×10⁻² s-two orders of magnitude greater than the dissociation of T-CEP binding to CD28. The binding to CD366 had a lower K_{D} (higher affinity), higher association rate (kₐ), and a faster dissociation rate relative to anti-CD3 mAbs binding to CD3εδ.²⁷ A relatively fast k_{off} represents a key factor in efficiently activating T cells, as it contributes to the turnover of TCR-MHC-peptide interactions.²⁸

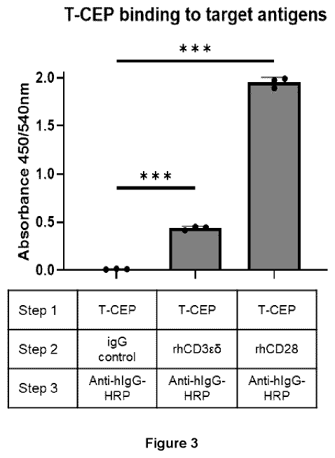

Binding to both targets simultaneously was analyzed using an enzyme-linked immunosorbent assay (ELISA). Consistent with the SPR results, both recombinant proteins bound T-CEP alone as evidence by a positive signal for Fc-tagged recombinant CD366 and CD28 individually, but absent in the case of an Fc-matched control (FIG. 3). To determine whether T-CEP could bind both targets simultaneously, T-CEP was first captured using immobilized recombinant CD366 then detected using recombinant CD28. CD365-Fc or the Fc control were first coated onto an ELISA plate and T-CEP subsequently added to wells, followed by incubation with biotinylated human CD28-Fc extracellular domain and detection with streptavidin-HRP. A positive signal was only observed in wells which contained both CD365-Fc and T-CEP, indicating that T-CEP is able to bind both targets at once. In contrast, wells coated with an Fc control or lacking T-CEP did not bind biotinylated CD28-Fc (FIG. 2B).

### Functional Characterization of T-CEP on Human T Cells

T-CEP Induces the Proliferation of Both Human CD4⁺ and CD8⁺ T Cells at Very Low Concentrations

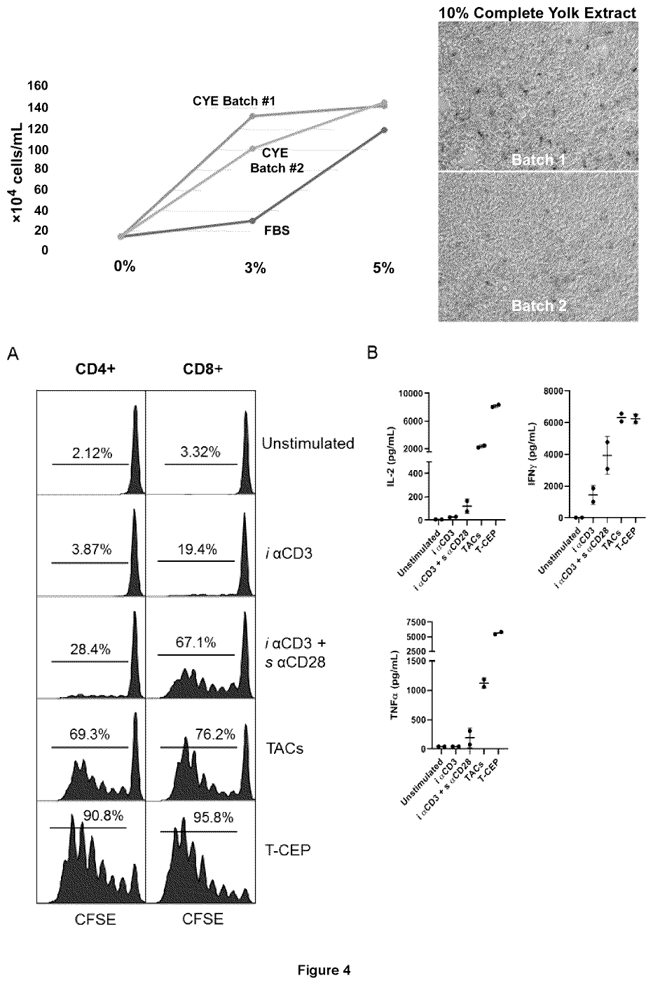

In order to determine the optimal working concentration of T-CEP, T cells isolated from human PBMCs were stained with carboxyfluorescein succinimidyl ester (CFSE; a cell proliferation tracer) and stimulated with T-CEP for 5 days over a range of T-CEP concentrations (50 μg/mL up to 10 μg/mL). Their proliferative activity, as evidence by a loss in CFSE signal, was analyzed by flow (results not shown). Comparable T cell proliferative activity by T-CEP was noted for concentrations as low as 500 μg/mL, however for all subsequent studies, a T-CEP concentration of 10 ng/mL (170 fM) was selected as it consistently yielded maximal T cell proliferation (FIG. 4A). Interestingly, the concentration of soluble T-CEP required to cause a full expansion of human T cells ex vivo was 150-fold less (in g/L) than required for TACs (when used per manufacturer instruction, ˜1.5 μg/mL, ˜2.5 pM). This T-CEP concentration was also far less than the optimal doses of immobilized αCD3 (i αCD3) (10 μg/mL) and soluble αCD28 (s αCD28) (1 μg/mL) mAbs. As expected, unstimulated T cells did not proliferate while anti-CD3 without co-stimulation led to a much-reduced level of both CD4⁺ and CD8⁺ human T cell proliferation.

Cytokine levels released in the medium by activated human CD3⁺ T cells were measured at Day 5 (FIG. 3B). Both T-CEP (10 ng/mL) and TACs (˜1.5 ug/mL) induced the production of IL-2, IFNγ and TNFα at higher levels than when T cells were stimulated with immobilized αCD3/soluble αCD28 mAbs.

### T-CEP Leads to the Activation and Expansion of T Cells Over the Course of 12 Days

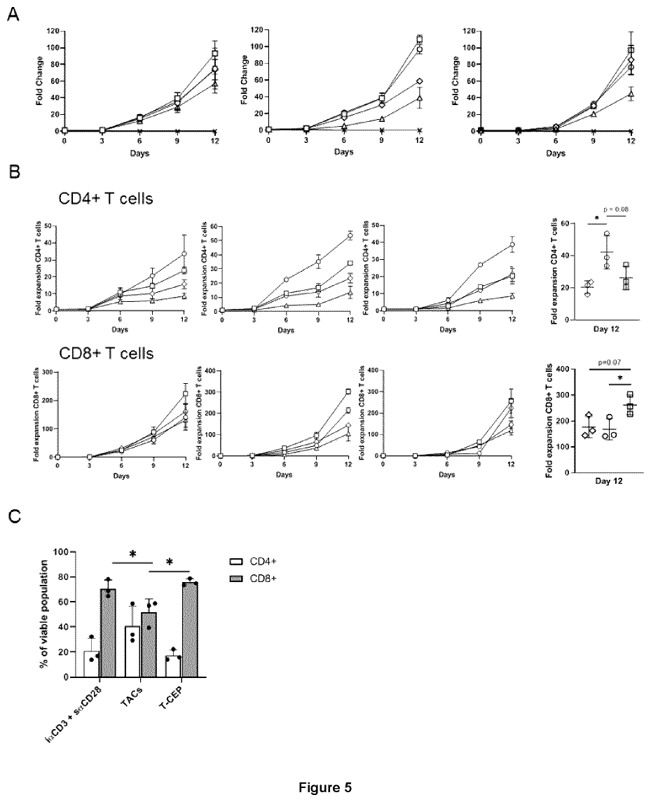

T cells used for adoptive cell therapy are required to undergo a rapid expansion phase lasting 12-14 days prior to re-infusion into the patient.¹ Human CD3⁺ T cells isolated from PBMCs of three healthy donors were expanded over the course of 12 days after a single stimulation, with IL-2 being added to the medium to further aid in cell expansion and activation ex vivo. Cells were transferred to G-Rex plates 3 days into the 12-day expansion stage. Every subsequent 3 days, aliquots were taken from duplicate wells to record the number of viable T cells present. T cells from the 3 donors were stimulated with T-CEP (10 ng/mL; 170 fM) or soluble TACs (25 μL/mL, ˜1.5 ug/mL), or with i αCD3 with or without s αCD28 (FIG. 5A). For all three donors, the highest levels of total T cell expansion were observed when T cells were treated ex vivo with T-CEP and TACs. As expected, unstimulated T cells did not expand, while T cells treated with i αCD3 alone displayed significantly less T cell expansion than the methods involving CD28 stimulation.

T cells were further analyzed by flow cytometry to monitor the expansion of CD4⁺ and CD8⁺ T cell subsets over 12 days for the 3 donors, relative to the starting number of CD4⁺ or CD8⁺ T cells before stimulation. Soluble TACs proved to be most effective at yielding higher numbers of CD4⁺ T cells as compared to T-CEP. In contrast, T-CEP favored the expansion of CD8⁺ T cells, yielding the highest number of CD8⁺ T cells, as compared to TACs. T-CEP resulted in a 237-fold mean expansion of human CD8⁺ T cells (Day 12); a level of CD8⁺ T cells expansion that was significantly higher than for CD8⁺ cells expanded with TACs (P<0.05) (FIG. 5B).

To further assess the extent of CD4⁺ and CD8⁺ T cell subsets expansion after 12 days under each expansion condition, we determined by flow cytometry the percentages of CD4⁺ and CD8⁺ T cell populations relative to the total population of viable lymphocytes recovered from the 3 donors. Based on percentage, T-CEP favored the expansion of CD8⁺ T cells over CD4⁺ T cells at a similar ratio of T cells produced by using a conventional method of i αCD3 with s αCD28. In contrast, TACs treatment led to a significantly reduced percentage of CD8⁺ T cells (P<0.05) (FIG. 5C).

Before stimulation, the CD4:CD8 ratios of T cells purified from healthy donor PBMCs were 2.1, 2.3, and 2.7 respectively.

The levels of activation markers CD25 and CD38 on human CD4⁺ and CD8⁺ T cells from the 3 donors were monitored by flow cytometry at specific time points during the 12-day expansion period. The increase in median fluorescence intensities (MFI) of CD25 expression peaked on Day 3, and on Day 6 for CD38. T-CEP induced the highest level of activation markers in the CD8⁺ and CD4⁺ T cells. However, the difference was not significant based on three donors. The MFI values then dropped by Day 12 to levels comparable to that before stimulation, irrelevantly of the expansion method used or the activation marker being monitored (FIG. 5A).

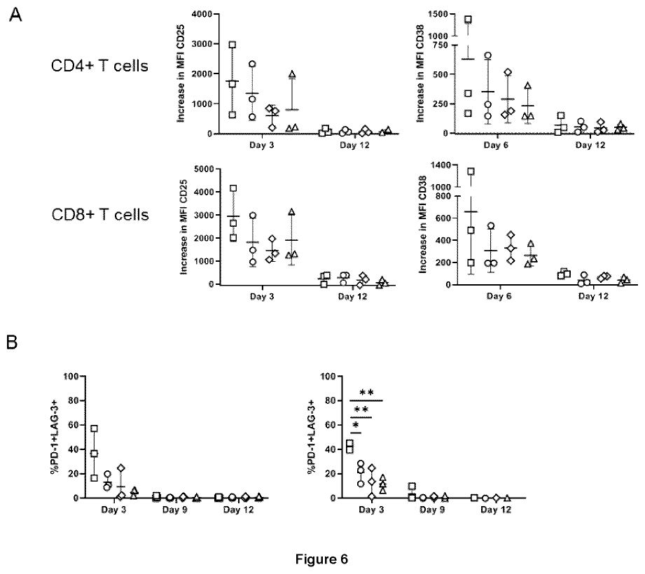

The surface expression of exhaustion markers PD-1 and LAG-3 co-expressed on CD4⁺ and CD8⁺ T cells was analyzed by flow cytometry for each expansion conditions (FIG. 6B). The percentage of CD4⁺ and CD8⁺ T cells co-expressing these markers peaked on Day 3 for all expansion methods tested, with a significantly higher level of co-expression in CD8⁺ T cells stimulated with T-CEP. However, this expression pattern returned to pre-activation levels by Day 9 and Day 12 in each stimulation method.

Stimulation with T-CEP Leads to Distinct Human T Cell Phenotypic Differentiation

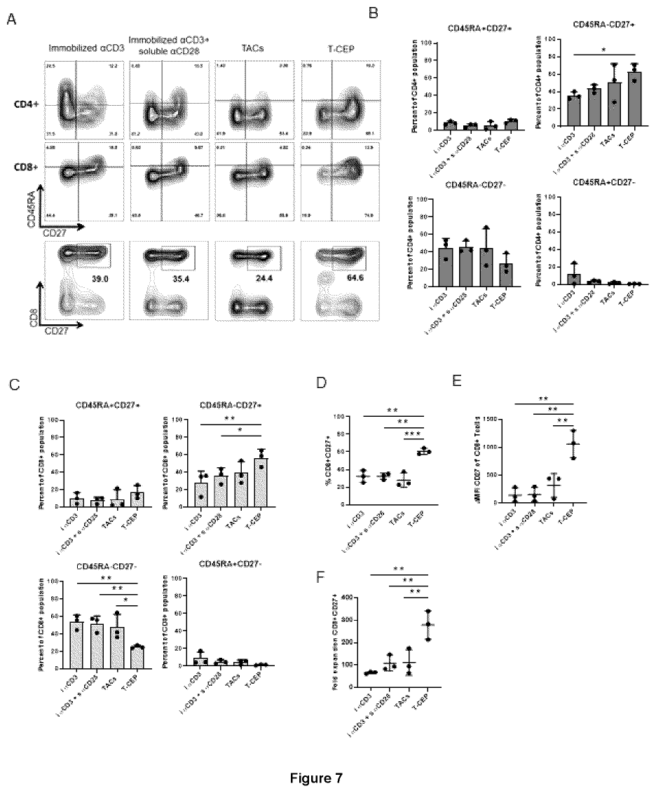

The differentiation patterns of human T cells from 3 donors were analyzed for the 12-day period. In particular, the expression levels of CD45RA and CD27 were monitored on CD4⁺ and CD8⁺ cells to provide an estimate of the differentiation state of these T cell subsets (FIG. 7A). Specifically, CD27 plays a role in T cell activation and differentiation, and its expression has been associated with in vivo persistence of T cells^{2,29}, while the loss of CD27 expression generally indicates late or terminal differentiation.³⁰ Meanwhile, CD45RA is expressed on early Tn and Tscm cell subsets, and its expression is lost upon differentiation into Tcm and Tem cells. This marker is then re-expressed upon differentiation into a late stage Temra subset.^{31,32} Therefore, it is expected that from least to furthest differentiated T cell subsets would display the following expression pattern of these 2 markers as follows: CD45RA⁺ CD27⁺<CD45RA-CD27⁺<CD45RA-CD27⁻<CD45RA+CD27⁻. After 12 days of expansion ex vivo, the T-CEP-treated human CD4⁺ T cell population in each donor, exhibited a low percentage of CD45RA-CD27⁻ and CD45RA+CD27⁻ cells and an increase in CD45RA-CD27⁺ T cells relative to T cells treated with i αCD3 alone (P<0.05) (FIG. 6B). Larger phenotypic changes were observed in the T-CEP-treated CD8⁺ T cell population, where the more differentiated CD45RA-CD27⁻ T cell population was significantly reduced to a mean (3 donors) of 25.2% of total CD8⁺ T cells in comparison to TACs (47.4%, P<0.05), i αCD3 with s αCD28 (51.5%, P<0.005) or i αCD3 alone (53.6%, P<0.005) (FIG. 7C.) In contrast, the less differentiated CD45RA-CD27⁺ T cell subset was significantly increased in T cells treated with T-CEP for 12 days with a (mean value, 56.5%) of the CD3⁺ CD8⁺ T cells, when compared to cells treated with i αCD3 with s αCD28 (35.9%, P<0.05) or i αCD3 alone (27.6%, P<0.005) of the cells. (FIG. 7C).

The number of transferred CD8⁺ CD27⁺ T cells has previously been associated with an improved patient response to the adoptive therapy treatment using autologous TILs for metastatic melanoma.² Stimulation with T-CEP increases the amount of CD8⁺ CD27⁺ T cells relative to the other methods of stimulation for each of the 3 donors (FIG. 6D) with a mean frequency of CD8⁺ CD27⁺ cells representing 60.8% of the viable lymphocyte population on Day 12. This percentage was significantly greater than the proportion of such cells observed under other expansion conditions (i αCD3, 32.2%, p=0.002; iαCD3 and s αCD28, 32.4%, P<0.005; TACs, 28.23%, P<0.005). This elevated population of CD8⁺ CD27⁺ T cells upon T cell expansion with T-CEP was independent of the T-CEP concentration over a 10 ng/mL to 10 μg/mL range (54-65% of total viable lymphocyte population) which contrast with TACs (range of 23%-38%) (data not shown). The Day 12 expression of CD27 was significantly higher in the T-CEP-treated group, as demonstrated by the greater ΔMFI (change in median fluorescent intensity) of CD27 relative to T cell groups treated with either i αCD3 (P<0.001), iαCD3 and s αCD28 (P<0.005), or with TACs (26.7%, P<0.01) (FIG. 7E). The fold expansion of CD8⁺ CD27⁺ T cells by Day 12 for each donor (n=3) was calculated using the percentage of CD8⁺ CD27⁺ cells derived from viable cells and the average total T cell count, relative to each donor initial CD8⁺ CD27⁺ T cell count. On average, T-CEP was found to increase the CD8⁺ CD27⁺ T cell count by 279 fold over 12 days in comparison to i αCD3 (67 fold, P<0.005), i αCD3 and s αCD28 (108 fold, P<0.05), or TACs (112 fold, P<0.05) (FIG. 7F).

### REFERENCES

1. Met, Ö., Jensen, K. M., Chamberlain, C. A., Donia, M. & Svane, I. M. Principles of adoptive T cell therapy in cancer. Seminars in Immunopathology 41, 49-58 (2019).
2. Rosenberg, S. A. et al. Durable complete responses in heavily pretreated patients with metastatic melanoma using T-cell transfer immunotherapy. Cancer Ther. 17, 4550-4557 (2011).
3. Zacharakis, N. et al. Immune recognition of somatic mutations leading to complete durable regression in metastatic breast cancer. Nat. Med. 24, 724-730 (2018).
4. Holstein, S. A. & Lunning, M. A. CAR T-Cell therapy in hematologic malignancies: A voyage in progress. Clin. Pharmacol. Ther. 107, 112-122 (2020).
5. Rosenberg, S. A., Restifo, N. P., Yang, J. C., Morgan, R. A. & Dudley, M. E. Adoptive cell transfer: a clinical path to effective cancer immunotherapy. Nat. Rev. Cancer 8, 299-308 (2008).
6. Phan, G. Q. & Rosenberg, S. A. Adoptive cell transfer for patients with metastatic melanoma: The potential and promise of cancer immunotherapy. Cancer Control 20, 289-297 (2013).
7. Li, D. et al. Genetically engineered T cells for cancer immunotherapy. Signal Transduct. Target. Ther. 4, 1-17 (2019).
8. Smith-Garvin, J. E., Koretzky, G. A. & Jordan, M. S. T cell activation. Annu. Rev. Immunol. 27, 591-619 (2009).
9. Jiang, X. et al. Adoptive CD8⁺ T cell therapy against cancer: Challenges and opportunities. Cancer Lett. 462, 23-32 (2019).
10. Li, K. et al. Adoptive cell therapy with CD4⁺ T helper 1 cells and CD8⁺ cytotoxic T cells enhances complete rejection of an established tumour, leading to generation of endogenous memory responses to non-targeted tumour epitopes. Clin. Transl. Immunol. 6, e160 (2017).
11. Gattinoni, L. et al. Acquisition of full effector function in vitro paradoxically impairs the in vivo antitumor efficacy of adoptively transferred CD8⁺ T cells. J. Clin. Invest. 115, 1467 (2005).
12. Crompton, J. G., Madhusudhanan, S. & Restifo, N. P. Uncoupling T-cell expansion from effector differentiation in cell-based immunotherapy. Immunol Rev 257, 264-276 (2014).
13. Klebanoff, C. A., Gattinoni, L. & Restifo, N. P. Sorting through subsets: Which T cell populations mediate highly effective adoptive immunotherapy?J immunother 35, 651-660 (2012).
14. Gattinoni, L., Klebanoff, C. A. & Restifo, N. P. Paths to sternness: building the ultimate antitumour T cell. Nat. Rev. Cancer 12, 671-84 (2012).
15. Petersen, C. T. et al. Improving T-cell expansion and function for adoptive T-cell therapy using ex vivo treatment with PI3Kd inhibitors and VIP antagonists. Blood Adv. 2, 210-223 (2018).
16. Li, Y. & Kurlander, R. J. Comparison of anti-CD3 and anti-CD28-coated beads with soluble anti-CD3 for expanding human T cells: Differing impact on CD8 T cell phenotype and responsiveness to restimulation. J. Transl. Med. 8, 104 (2010).
17. Kagoya, Y. et al. Transient stimulation expnds superior antitumor T cells for adoptive therapy. JCI insight 2, 89580 (2017).
18. Giannoni, F. et al. Clustering of T Cell ligands on artificial APC membranes influences T Cell activation and Protein Kinase C θ translocation to the T cell plasma membrane. J. Immunol. 174, 3204-3211 (2005).
19. O'Connor, R. et al. Activation and Proliferation Substrate Rigidity Regulates Human T Cell. J Immunol 189, 1330-1339 (2012).
20. Judokusumo, E., Tabdanov, E., Kumari, S., Dustin, M. L. & Kam, L. C. Mechanosensing in T lymphocyte activation. Biophys. J. 102, L5-L7 (2012).
21. Bortoletto, N., Scotet, E., Yoichi, M., D'Oro, U. & Lanzavecchia, A. Optimizing anti-CD3 affinity for effective T cell targeting against tumor cells. Eur. J. Immunol. 32, 3102-3107 (2002).
22. Lanzavecchia, A. & Sallusto, F. Understanding the generation and function of memory T cell subsets. Curr. Opin. Immunol. 17, 326-332 (2005).
23. Löffler, A. et al. A recombinant bispecific single-chain antibody, CD19 X CD3, induces rapid and high lymphoma-directed cytotoxicity by unstimulated T lymphocytes. Blood 95, 2098-2103 (2000).
24. Grosse-Hovest, L. et al. A recombinant bispecific single-chain antibody induces targeted, supra-agonistic CD28-stimulation and tumor cell killing. Eur. J. Immunol. 33, 1334-1340 (2003).
25. Chen, X., Zaro, J. L. & Shen, W. C. Fusion protein linkers: Property, design and functionality. Adv. Drug Deliv. Rev. 65, 1357-1369 (2013).
26. Isamu, A. & KOKAJI. Soluble Antibody Complexes for T cell or NK cell Activation and Expansion. (2015).
27. Law, C.-L. et al. Expression and characterization of recombinant soluble human CD3 molecules: presentation of antigenic epitopes defined on the native TCR-CD3 complex. Int. Immunol. 14, 389-400 (2002).
28. Dushek, O. et al. Antigen potency and maximal efficacy reveal a mechanism of efficient T cell activation. Sci. Signal. 4, ra39 (2011).
29. Powell, D. J., Dudley, M. E., Robbins, P. F. & Rosenberg, S. A. Transition of late-stage effector T cells to CD27+CD28+ tumor-reactive effector memory T cells in humans after adoptive cell transfer therapy. Blood 105, 241-250 (2005).
30. Hamann, D. et al. Evidence that human CD8+CD45RA+CD27⁻ cells are induced by antigen and evolve through extensive rounds of division. Int. Immunol. 11, 1027-1033 (1999).
31. Larbi, A. & Fulop, T. From ‘truly naïve’ to ‘exhausted senescent’ T cells: When markers predict functionality. Cytom. Part A 85, 25-35 (2013).
32. Di Mitri, D. et al. Reversible senescence in human CD4+CD27+CD45RA+ memory T cells. J. Immunol. 187, 2093-2100 (2011).
33. Rabenstein, H. et al. Differential kinetics of antigen dependency of CD4+ and CD8+ T cells. J. Immunol. 192, 3507-3517 (2014).
34. Foulds, K. E. et al. Cutting edge: CD4 and CD8 T Cells are intrinsically different in their proliferative responses. J Immunol Ref. 168, 1528-1532 (2002).
35. Kaartinen, T. et al. Low interleukin-2 concentration favors generation of early memory T cells over effector phenotypes during chimeric antigen receptor T-cell expansion. Cytotherapy 19, 689-702 (2017).
36. Prieto, P. A., Durflinger, K. H., Wunderlich, J. R., Rosenberg, S. A. & Dudley, M. E. Enrichment of CD8+ cells from melanoma tumor-infiltrating lymphocyte cultures reveals tumor reactivity for use in adoptive cell therapy. J. Immunother. 33, 547-556 (2010).
37. Klebanoff, C. A. et al. Central memory self/tumor-reactive CD8+ T cells confer superior antitumor immunity compared with effector memory T cells. Proc. Natl. Acad. Sci. 102, 9571-9576 (2005).
38. Sommermeyer, D. et al. Chimeric antigen receptor-modified T cells derived from defined CD8+ and CD4+ subsets confer superior antitumor reactivity in vivo. Leukemia 30, 492-500 (2016).
39. Fritsch, R. D. et al. Stepwise differentiation of CD4 memory T cells defined by expression of CCR7 and CD27. J. Immunol. 175, 6489-6497 (2005).
40. Mahnke, Y. D., Brodie, T. M., Sallusto, F., Roederer, M. & Lugli, E. The who's who of T-cell differentiation: Human memory T-cell subsets. Eur. J. Immunol. 43, 2797-2809 (2013).
41. Hamann, D. et al. Phenotypic and functional separation of memory and effector human CD8+ T cells. J. Exp. Med. 186, 1407-1418 (1997).
42. Schiött, Â., Lindstedt, M., Johansson-Lindbom, B., Roggen, E. & Borrebaeck, C. A. K. CD27-CD4+ memory T cells define a differentiated memory population at both the functional and transcriptional levels. Immunology 113, 363-370 (2004).
43. Callender, L. A. et al. Human CD8+ EMRA T cells display a senescence-associated secretory phenotype regulated by p38 MAPK. Aging Cell 17, e12675 (2018).
44. Graef, P. et al. Serial transfer of single-cell-derived immunocompetence reveals stemness of CD8+ central memory T cells. Immunity 41, 116-126 (2014).
45. Huang, J. et al. Modulation by IL-2 of CD70 and CD27 expression on CD8⁺ T cells: importance for the therapeutic effectiveness of cell transfer immunotherapy. J. Immunol. 176, 7726-35 (2006).
46. Cohen, A. D. et al. B cell maturation antigen-specific CAR T cells are clinically active in multiple myeloma. J. Clin. Invest. 129, 2210-2221 (2019).
47. Hendriks, J. et al. CD27 is required for generation and long-term maintenance of T cell immunity. Nat. Immunol. 1, 433-440 (2000).
48. Hendriks, J., Xiao, Y. & Borst, J. CD27 promotes survival of activated T cells and complements CD28 in generation and establishment of the effector T cell pool. J. Exp. Med. 198, 1369-1380 (2003).
49. Neeson, P. et al. Ex vivo culture of chimeric antigen receptor T cells generates functional CD8 T cells with effector and central memory-like phenotype. Gene Ther. 17, 1105-1116 (2010).
50. Cha, E., Graham, L., Manjili, M. H. & Bear, H. D. IL-7+IL-15 are superior to IL-2 for the ex vivo expansion of 4T1 mammary carcinoma-specific T cells with greater efficacy against tumors in vivo. Breast Cancer Res. Treat. 122, 359-369 (2010).
51. Prodeus, A. et al. VISTA.COMP—an engineered checkpoint receptor agonist that potently suppresses T cell-mediated immune responses. JCI Insight 2, e94308 (2017).

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A bispecific scFv construct agonizing both CD3 and CD28 pathways.

2. The construct of claim 1, wherein the construct is soluble.

3. The construct of claim 1 or 2, wherein the construct activates, expands, and differentiates human T cells ex vivo.

4. The construct of claim 3, wherein the construct is active at concentrations in the femtomolar range, such as from about 10 to about 500 fM, such as about 170 fM.

5. The construct of claim 1, wherein the construct promotes the preferential growth of human CD8⁺ T cells over the course of 12 days in comparison to methods involving immobilized anti-CD3 mAb/soluble anti-CD28 mAb or soluble anti CD3/CD28 mAb complexes.

6. The construct of claim 1, wherein the construct favors the expansion of a CD8⁺ CD27⁺ T cell phenotype.

7. The construct of claim 1, wherein (i) the anti-CD28 scFv is at the N-terminus of the construct and the anti-CD3 scFv is at the C-terminus of the construct or ii) the anti-CD3 scFv is at the N-terminus of the construct and the anti-CD28 scFv is at the C-terminus of the construct.

8. (canceled)

9. The construct of claim 1, comprising one or more flexible linkers, such as one, two, or three flexible linkers.

10. The construct of claim 9, comprising a flexible linker between each heavy and light chain domain of each scFv as well as a flexible linker between each scFv.

11. The construct of claim 1, wherein the construct engages both signals for TCR activation and co-stimulation at a molar ratio of 1:1.

12. The construct of any one of claims 1 to 11, comprising a purification and/or detection tag.

13. The construct of claim 12, comprising a histidine tag.

14. The construct of claim 1, comprising or consisting of a polypeptide having at least 80% sequence identity to SEQ ID NO:1:
DIVLTQSPASLAVSLGQRATISCRASESVEYYVTS
LMQWYQQKPGQPPKLLIFAASNVESGVPARFSGSG
SGTNFSLNIHPVDEDDVAMYFCQQSRKVPYTFGGG
TKLEIKRGGGGSGGGGSGGGGSQVKLQQSGPGLVT
PSQSLSITCTVSGFSLSDYGVHWRQSPGQGLEWLG
VIWAGGGTNYNSALMSRKSISKDNSKSQVFLKMNS
LQADDTAVYYCARDKGYSYYYSMDYWGQGTTVTVS
SASTKGPSVFPLAPSSGSGGGGSGGGGSGGGGSDI
KLQQSGAELARPGASVKMSCKTSGYTFTRYTMHWW
KQRPGQGLEWIGYINPSRGYTNYNQKFKDKATLTT
DKSSSTAYMQLSSLTSEDSAVYYCARYYDDHYCLD
YWGQGTTLTVSSVEGGSGGSGGSGGSGGVDDIQLT
QSPAIMSASPGEKVTMTCRASSSVSYMNWYQQKSG
TSPKRWIYDTSKVASVPYRFSGSGSGTSYSLTISS
MEAEDAATYYCQQWSSNPLTFGAGTKLELKHHHHH
H
or a fragment thereof.

15. The construct of claim 14, comprising or consisting of a polypeptide having at least 85, 90, 95, 96, 97, 98, or 99% identity to SEQ ID NO:1, or a fragment thereof.

16. The construct of claim 14, comprising or consisting of a polypeptide having SEQ ID NO:1.

17. A polynucleotide encoding the construct of claim 1.

18. The polynucleotide of claim 17, comprising or consisting of a polynucleotide having at least 80% sequence identity to SEQ ID NO:2:
gacatcgtgctgacacagagccctgcttctctggc
cgtgtctctgggacagagagccaccatcagctgta
gagccagcgagagcgtggaatattacgtgaccagc
ctgatgcagtggtatcagcagaagcctggccagcc
tcctaagctgctgatcttcgccgccagcaatgtgg
aaagcggagtgcctgccagattttccggctctggc
agcggcaccaacttcagcctgaacattcaccccgt
ggacgaggacgacgtggccatgtacttttgccagc
agagcagaaaggtgccctacacctttggcggaggc
accaagctggaaatcaagagaggtggcggaggatc
tggcggcggaggaagcggaggcggcggatctcaag
tgaaactgcagcagtctggccctggcctggtcaca
ccttctcagagcctgagcatcacctgtaccgtgtc
cggctttagcctgagcgattacggcgtgcactggg
tccgacagtctccaggacaaggactggaatggctg
ggagtgatttgggctggcggagggacaaactacaa
cagcgccctgatgagccggaagtccatcagcaagg
acaacagcaagagccaggtgttcctgaagatgaac
tccctgcaggccgacgacaccgccgtgtactattg
cgccagagacaagggctacagctactactacagca
tggactactggggccagggcaccaccgtgacagtt
agctctgcctctacaaagggccccagcgtgttccc
tctggctccttctagttctggaagtggcggtggtg
gatcaggcggtggcggttctggcggaggcggaagt
gatattaagctgcagcagagcggagccgagctggc
tagacctggtgcctctgtgaagatgagctgcaaga
ccagcggctacaccttcaccagatacaccatgcat
tgggtcaagcagcggcctggacagggacttgagtg
gatcggctacatcaaccccagccggggctacacca
actacaaccagaagttcaaggacaaggccacactg
accaccgacaagagcagcagcacagcctacatgca
gctgagcagcctgaccagcgaagatagcgccgtgt
attactgtgcccggtactacgacgaccactactgc
ctggattattggggacagggaacaaccctgaccgt
gtctagtgtggaaggtggcagtggcggtagcggtg
gctctggtggaagcggcggagtggatgatatccag
ctgactcagtcccctgccatcatgtctgctagccc
tggcgagaaagtgaccatgacctgcagagccagca
gctccgtgtcctacatgaactggtatcaacaaaag
agcggcacaagccccaagcggtggatctacgatac
aagcaaggtggccagcggcgtgccctatagatttt
ctggaagcggatccggcaccagctactccctgaca
atcagcagcatggaagccgaggatgccgccaccta
ctactgccaacagtggtccagcaatcccctgacct
ttggagccggaacaaagctggaactgaagcaccac
caccatcaccac
or a fragment thereof.

19. (canceled)

20. (canceled)

21. (canceled)

22. (canceled)

23. (canceled)

24. (canceled)

25. A method for expanding, activating, and or differentiating T cells ex vivo, the method comprising incubating the T cells with the construct of claim 1.

26. The method of claim 25, wherein the construct is used at concentration of from about 10 to about 500 fM, such as about 170 fM.

27. The method of claim 25, wherein the incubating is for a period of about 1, 2, 3, 4, 5, 6, 7, 8, 9, 10, 11, 12, 13, or 14 days, such as about 12 days.

28. (canceled)

29. (canceled)
